(19) United States
(12) Patent Application Publication
(10) Pub. No.: US 2022/0015648 A1
(43) Pub. Date: Jan. 20, 2022

(54) MONITORING DEVICE, SENSOR DEVICE, AND SYSTEM AND METHOD FOR MONITORING A PERSON

(71) Applicant: DOKOKI AG, Hindelbank (CH)

(72) Inventor: Marc ZINNER, Mühlethal (CH)

(21) Appl. No.: 17/293,803

(22) PCT Filed: Nov. 13, 2019
(86) PCT No.: PCT/EP2019/081211

§ 371 Date: May 13, 2021

(30) Foreign Application Priority Data
Nov. 13, 2018 (DE) 10 2018 128 463.2

(51) Int. Cl. A61B 5/0205; H04N 5/232; H04R 1/02; H04R 1/08; G06F 3/14; G08B 5/36; G08B 21/02; H04W 4/38; H02J 7/00; A61B 5/024; A61B 5/1455; A61B 5/00; A61B 5/021; A61B 5/145

(52) U.S. Cl. CPC A61B 5/02055; A61B 2503/04; H04R 1/028; H04R 1/08; G06F 3/14; G08B 5/36; G08B 21/0277; G08B 21/0211; H04W 4/38; H02J 7/0048; H02J 7/0045; A61B 5/02438; A61B 5/14551; A61B 5/742; A61B 5/7405; A61B 5/0022; A61B 5/6804; A61B 5/7455; A61B 5/021; A61B 5/14532; A61B 2562/0204; A61B 2560/0456; H04N 5/23299

(57) ABSTRACT

A monitoring device for a person, in particular for monitoring a sleeping infant, comprising a base station and a sensor device that is able to be connected to an item of clothing of the person, wherein the sensor device is connected wirelessly to the base station to transmit the vital data measured by the sensors, wherein the monitoring module of the base station comprises a camera that is able to be oriented towards the person by pivoting the monitoring module with respect to the base, wherein the monitoring module comprises an input unit and a display unit. A monitoring device for a person or a sensor device for use in a monitoring device or a method for monitoring a person that makes it possible to reliably monitor the person and important vital data of the person.

The invention relates to a monitoring device for a person, in particular for monitoring a sleeping infant. The invention also relates to a sensor device for use in such a monitoring device. Finally, the invention also relates to a system and a method for monitoring a person, in particular for monitoring a sleeping infant.

In practice, various monitoring devices for sleeping infants have become known, which are also known colloquially as “baby monitors.” By means of a built-in microphone, these wirelessly transmit sounds, in particular cries from the infant, via a loudspeaker to a second device, the two most commonly being connected by radio. The disadvantage in this case is that although it is possible to reliably detect when an infant wakes up and cries, continuous monitoring of vital data of the infant is not possible.

In practice, monitoring devices for small children are also known in which a camera is integrated that shows an image of a small child on an external display. In this case as well, there is the disadvantage that important vital data are neither reliably captured nor processed in a meaningful way or clearly presented.

Devices have also become known from practice in which a sensor device that can be connected to the item of clothing of a person who plays sports, for example, captures vital data of the athlete and stores it in a smartphone, for example. Although the vital data can be analyzed easily in retrospect, such a sensor device is not suitable for small children, since the children move in their sleep and there is therefore the risk that the sensor device will be removed or impaired.

Sensor devices are also known from practice, such as are used for medical devices, in which the sensors are connected to an evaluation device by cable. However, the corresponding connection is not suitable for monitoring the sleep of an infant, since the cables easily tear off when the infant moves.

U.S. Pat. No. 2,015,015 72 63 A1 describes a monitoring device for a person, comprising a base station that has a monitoring module; and a sensor device that can be connected to an item of clothing of the person in the form of a sock. In this case, the sensor device comprises a power source that can be charged several times and that supplies the sensor device with power. The sensor device comprises a sensor for measuring the oxygen saturation in the blood and for detecting the heartbeat or the pulse of the person, using the principle of pulse oximetry. A charging station with inductive charging of the sensor device is proposed. In use, the sensor device is wirelessly connected to the base station for transmitting the measured vital data. It is also proposed to arrange an external camera in the room with the person, which can also be pointed at the person. The base station also has display units and an input unit. A disadvantage of the known monitoring device is, on the one hand, that the measurement of the oxygen saturation according to the principle of pulse oximetry is susceptible to interference and is only possible at a few places on the body that allow light transmission. Furthermore, the measurement options on the foot are very limited. The sensor device does not have a housing and cannot be detached from the item of clothing without being destroyed; it is incorporated permanently into the sock or ankle cuff. As such, the sock cannot be washed and must be used as a disposable item for hygienic reasons. In order to press the sensors against the foot, the ankle cuff must be placed tightly on the foot, which can impair blood circulation. Furthermore, the monitoring module does not allow the reproduction of captured images, and thus a verification of the function of the overall system.

DE 10 2011 077 515 A1 describes a monitoring device for a person, comprising a base station that can be supplied with power, which has a monitoring module with a display unit, which are accommodated in a common housing, and a sensor device that can be connected to an item of clothing of the person. The sensor device comprises a rechargeable power source that supplies the sensor device with power, the sensor device comprising a plurality of sensors, including a sensor for detecting the heartbeat. The base station includes a charging station for the rechargeable power source. The sensor device is wirelessly connected to the base station for transmitting the vital data captured by the sensors. The known monitoring device has the disadvantage that no commands can be entered via the monitoring device and no images of the person can be displayed. Furthermore, the sensor device is unsuitable for connection to an item of clothing and is therefore preferably designed as a disposable plaster.

U.S. Pat. No. 2,016,028 70 74 A1 describes a monitoring device for a person, comprising a base station and a sensor device that can be connected to a bracelet for the person, the sensor device comprising a housing and a rechargeable power source that supplies the sensor device with power, wherein the sensor device comprises a plurality of sensors and can be connected to an external charging station for charging the power source. In this case, the sensor device is connected to the base station for transmitting the measured vital data. The monitoring module comprises a camera that captures an image of the person being monitored, and an audio sensor that captures acoustic signals. The disadvantage of the known monitoring device is the fact that the camera in the base station cannot be aimed at the person. Furthermore, no options are provided on the base station itself to display the captured image or to make inputs directly.

U.S. Pat. No. 2,015,010 94 41 A1 describes a monitoring device for monitoring a person, comprising a base station that can be supplied with power and that has a base and a monitoring module that can be pivoted with respect to the base, the monitoring module of the base station comprising a camera that, by pivoting the monitoring module with respect to the base, can be oriented towards a person about the axis formed by joints, the monitoring module comprising display units such as the status indicator. The monitoring device transmits wirelessly captured image data and acoustic data, which are captured via a microphone on the monitoring module, to external users. Furthermore, a loudspeaker is provided for playing music or for transmitting conversations or singing by an operator, as well as infrared light sources in order to be able to capture an image with the camera even in the dark. The disadvantage of the known monitoring device is that no vital parameters of the person being monitored are captured, that there is no possibility of displaying the captured data on the base station, and that inputs at the base station are not possible.

It is the object of the invention to provide a monitoring device for a person and/or a sensor device for use in a monitoring device, and/or a system and a method for monitoring a person, which enables reliably monitoring the person and important vital data of the person.

According to the invention, this object is achieved by a monitoring device and/or a sensor device and/or a system and method having the features of an independent claim.

According to an aspect of the invention, a monitoring device for a person, in particular for monitoring a sleeping infant, is created, comprising a base station that can be supplied with power and that has a base and a monitoring module that can be pivoted with respect to the base, and a sensor device that can be connected to an item of clothing of the person. The sensor device comprises a power source that can be recharged, such as a lithium ion battery, which supplies the sensor device with power. The sensor device furthermore comprises a sensor for measuring the oxygen saturation in the person's blood and a sensor for detecting the heartbeat or the pulse of the person, such that the important vital data of oxygen saturation and heartbeat can be reliably captured. The base station has a charging station for the rechargeable power source of the sensor device, such that it is not necessary to supply power to the sensor device or its rechargeable power source using a wire via the building power supply or another external power source. As a result, the base station also performs the function of the charging station for the sensor device. The sensor device is wirelessly connected to the base station for transmitting the vital data measured by the sensors of the sensor device, such that false alarms caused by a cable being tom off are reliably prevented. The monitoring module of the base station comprises a camera that can be pointed at the person being monitored by it pivoting with respect to the base, the camera being able to capture a series of individual images or a video stream. The monitoring module finally has an input unit and a display unit, which serve as user interfaces. The monitoring device distinguishes in that the input unit is designed as a curved, transparent pane with touch functionality, and in that the display unit is designed as a flat display arranged behind the transparent pane. As a result, the input unit can be used expediently for inputting commands and for operating the ic monitoring device, the curved pane being joined with the overall rounded contour of the monitoring module. The display is expediently arranged behind the transparent pane, which is preferably made of plastic or glass and is therefore largely scratch-resistant, the display unit making it possible to show the information for an operator, and/or to display films, photos, or similar graphic content that promotes the sleep of an infant. The monitoring module can thus be used similarly to a tablet PC, with the proviso that the input unit is curved. This ensures, in particular, that there are on the monitoring module no sharp-edged corners that could lead to injury to the person being monitored. The curved pane also facilitates capturing the image of the person being monitored.

The base station preferably comprises a microphone that captures acoustic signals, in particular those of the person being monitored, but also other sounds from the monitored room. The microphone is expediently built into the monitoring module and can accordingly be oriented, together with the camera, towards the person being monitored. A downstream evaluation and filter logic can eliminate certain acoustic signals or not transmit them to a supervising caregiver, for example snoring sounds, breathing, and the like. In contrast, certain code words, e.g. “help” or the like, can trigger an alarm. With a neural network, after a short learning phase, different types of infant or baby screams can be classified, expediently taking into account the vital data.

According to a further advantageous embodiment it is provided that the base station comprises a loudspeaker that can generate prespecifiable acoustic signals. The loudspeaker does not serve to reproduce the sounds captured by the microphone, because such an “echo function” makes little sense when monitoring a person; rather, lullabies or background sounds that match the display can be output via the loudspeaker. In particular, it is possible for the monitoring person to reply to the sounds captured by the microphone on another terminal, or to speak calming words. In the case of an adult patient, a verbal exchange can take place in this way, even with a caregiver positioned remotely.

The monitoring module expediently has at least one infrared bulb that illuminates the field of view of the camera and thus enables an image of the person being monitored to be transmitted even in the dark. It is possible to provide two or more infrared bulbs for this purpose.

According to an expedient refinement, it is provided that the base station comprises at least one lighting arrangement that is dimmable. This can be an LED or a plurality of LEDs that can change their colors and are adjustable in their light intensity. The dimmable lighting arrangement makes it easier for a person to fall asleep by initially leaving the light on and then gradually dimming it down until the lighting arrangement finally no longer emits light, which in particular can be used to prevent anxiety among small children in dark rooms.

The base station advantageously comprises LEDs of different colors to represent alarm states, for example an LED in green if all the captured vital parameters satisfy an “OK” condition, a yellow one if the captured vital parameters satisfy a problematic condition, and a red LED if the captured vital parameters meet a critical condition. As a result, even for a viewer who is not authorized to operate the monitoring device, it can be recognized at first glance whether or not the captured vital parameters require the expertise of a specially trained caregiver, such as a doctor.

According to a particularly expedient embodiment, it is provided that the base station is modeled on the silhouette of a small child. Such a silhouette has a calming and familiar effect on small children, in particular due to the comparatively large proportion of the head in relation to the torso of the silhouette. Here, the base corresponds to the trunk, including the arms and legs of the infant silhouette, and the monitoring module corresponds to the infant's head.

The eye area of the monitoring module corresponding to the head is expediently implemented here by the input unit, which is designed as a curved, transparent pane with touch functionality.

The monitoring module, that is to say the head shape, expediently carries a cap that can be designed in the manner of an elongated night cap and that preferably extends down to the floor and additionally, as a third pillar, prevents the base from tipping over. In this case, the cap can be equipped with an elastically deformable part that makes it possible to implement the third leg even when the monitoring module is inclined.

The base station is expediently supplied with power by alternating current, and has a charging station for the rechargeable power source of the sensor device. The charging station provides direct current for the power source of the sensor device, such that the sensor device applied to the item of clothing of the person being monitored cannot emit any voltages and/or currents that are hazardous to the body. This advantageously ensures that the sensor device does not have to be charged using a cable.

The rechargeable power source is expediently designed by inductive charging from the base station, such that it is possible to encapsulate the sensor device as a whole, and thus also to clean it using liquids, such that the increased hygiene requirements of a multi-use sensor device are met.

The charging station is expediently provided in the base, for example approximately in the middle on the front.

In a preferred embodiment, the sensor device can be inserted into the charging station with a clamping effect, such that the sensor device is held in the charging station in a defined position that facilitates inductive charging. Alternatively, contacts of the charging station can also be brought into connection in this way with contacts of the sensor device. The clamping of the sensor device in the base station makes it possible, in particular, to transport the combination of monitoring device and sensor device together, and at the same time to ensure that the sensor device can be reliably stowed and found again as a result of the defined location.

It is possible for more than one sensor device to be assigned to a monitoring device—for example if different vital parameters must be captured, or if the sensor devices will be alternated so that one captures the vital parameters while the other sensor device is being charged in the charging station. However, precisely one sensor device is preferably assigned to the monitoring device so that there is no confusion with neighboring monitoring devices when a person is being monitored.

The base station preferably has a display of the charge status of the rechargeable power source of the sensor device, which indicates to a user when charging of the sensor device is necessary, or for how long the sensor device can still capture vital data of the person being monitored.

According to an expedient implementation, it is provided that the base station is equipped in its foot with fixing means that allow the base station to be fixed to a surface. This advantageously ensures that the person being monitored cannot be lost from the field of view of the camera even in the event of vibrations, earthquakes or the like. According to a first possible embodiment it is provided that a magnet, for example a neodymium magnet, is provided in the foot area of the base, which magnet can be fixed on a magnetizable base. Alternatively, suction cups or the like can be contemplated. According to another implementation, screw holes can be formed in the foot of the base, which allow the foot to be connected to a surface, in particular by screws.

The base station expediently comprises a wireless transmitter, in particular a radio transmitter, wireless LAN transmitter, and/or a Bluetooth transmitter, which enables the transmission of data to an—external—receiving unit. The transmitted data include, in particular, the measured vital data or vital parameters, but also the acoustic and optical signals, such that it is possible to monitor the sounds, the image and the captured vital parameters from a remote device. For this purpose, the corresponding data are made available in a network. The receiving unit can, for example, be a smartphone of the parents, who then receive the current data of the person being monitored live.

The base station furthermore preferably comprises a receiver, in particular a radio receiver, wireless LAN receiver, and/or Bluetooth receiver, which in particular can receive control commands from the network. At the same time, the receiver wirelessly receives the vital parameters from the sensor device, and a processor is preferably provided in the base station, which processes the measurement data mathematically and graphically and preferably also stores it locally. It is possible to store the data in an internet cloud or on a central server that is optionally connected to the base station by wire, or wirelessly.

The sensor device expediently comprises a housing in which is arranged a rechargeable power source that supplies the sensor device with power. The housing is preferably completely encapsulated to protect against the ingress of liquid. This can be done, for example, by sealing all openings.

The housing preferably has a connection means for connection to an item of clothing of the person being monitored. The connection means is advantageously used to fix the sensor device to the item of clothing, for example by means of a push button or a magnetic fastener. It is also possible to connect the housing to the item of clothing by clamping. The housing of the sensor arrangement is expediently designed in the manner of a disk, in particular in the manner of a flat disk, wherein one of the faces of the disk that faces the person has the sensors, which can be brought into contact with contacts on the item of clothing, or with the person's body. The connection means preferably serve at the same time for electrical contact with an electrically conductive layer on the item of clothing of the person being monitored.

The sensor is expediently biased in the direction of the person being monitored by a spring arrangement, such that it can reliably capture the person's vital data. The bias by a spring arrangement ensures that the sensor rests on the body surface of the person and can thus capture the specific vital data on the body surface directly, without interference and with consistent quality. It is possible that the item of clothing is also equipped with sensor surfaces, which are expediently designed to be machine washable together with the item of clothing. The contact to these contacts is then advantageously an electrical contact point.

The sensor device advantageously comprises a transmission arrangement that wirelessly transmits the vital data captured by the sensors to an external receiving station. The external receiving station can be the base station of the monitoring device, or can be another station, for example a medical device or a user's tablet PC.

According to an aspect of the invention, a sensor device for use in a monitoring device as described above is created, comprising a housing in which a rechargeable power source is arranged, which supplies the sensor device with power; a connection means provided on the housing for an item of clothing of a person being monitored; a sensor that is biased in the direction of the person being monitored by a spring arrangement and that can detect the person's vital data; and a transmission arrangement that wirelessly transmits the vital data captured by the sensors to an external receiving station. It is also possible in this case to provide more than one sensor, since each of the vital signs require a specific sensor. The sensor device distinguishes in that the sensor is arranged on a lift board, in that the lift board is connected by a flexible board element to a first base board and a second base board, and in that the lift board can be moved into a raised position by means of the flexible board elements and the spring arrangement, by pushing the first base board and the second base board together. By arranging the sensor on a lift board that is supported on both ends by the flexible board element and that is electrically connected to the first base board and to the second base board, and also by providing the spring arrangement, it is advantageously achieved that the sensor is always pressed against the skin area of the person being monitored, even if the person changes their relative position with respect to the item of clothing in different sleeping positions, or due to nocturnal movement. As a result, the sensor and the lift board can follow the surface, which is not always flat, of the body of the person being monitored, and thus reliably capture the given vital data without interruption and with consistent quality. This means that false alarms are not triggered—or are triggered only very rarely.

The pushing together of the first base board and the second base board is expediently carried out in such a way that, for attachment to the first base board and the second base board, the lift board is arranged centrally between the two base boards and connected to them via the flexible board elements, and only when the sensor device is assembled are both base boards pushed together—for example, by pushing at least one base board in the direction of the other base board—in such a manner that the centrally arranged lift board has to move out of the way. The lift board in this case takes up approximately the area of a recess that is delimited by the two base boards, wherein the spring arrangement biases the lift board through the recess in the direction of deployment.

A first and a second sensor are expediently provided, the first sensor and the second sensor being arranged on two parallel, independently spring-loaded lift boards. As a result, the first sensor can follow a change in the contour of the person being monitored independently of the second sensor, such that the first sensor and the second sensor can each reliably capture the vital data on the surface of the person being monitored. It is also possible to provide three or more sensors, wherein in each case different sensors can be provided, and also redundant sensors can be provided to ensure the correctness of the measurement. In particular, it is possible that two or more different sensors can also be arranged on a lift board.

Each of the lift boards is advantageously connected to the base board with its own flexible board elements and thereby enables individual tracking to changed surfaces. Alternatively, a shared flexible board element can also be provided that connects several lift boards to a base board.

The sensor, or a sensor, is preferably selected from a group comprising: a sensor for measuring the oxygen saturation in the person's blood, a sensor for detecting the heartbeat or the pulse of the person, a sensor for detecting the surface body temperature of the person, a sensor for detecting the blood pressure of the person, a sensor for detecting blood sugar, and combinations thereof. In particular, based on the vital data captured by one or more sensors, it is also possible to derive complex information such as electrocardiograms, or other profiles over time. Based on the surface body temperature of the person, if the measuring point is known, the core body temperature of the person can be calculated, the fluctuation range of the calculation remaining within narrow limits due to the fixation on a piece of clothing. The oxygen saturation in the blood can, for example, be determined optically without a needle by detecting the reflectivity.

At least the oxygen saturation in the person's blood is expediently measured by a corresponding sensor, because the oxygen saturation in the blood is an indicator of the person's inhalation function and thus a reliable early indicator of disturbances in the person's vital functions. Furthermore, a sensor is preferably provided for detecting the heartbeat or the pulse, since the heartbeat or changes in the heartbeat are also a reliable indicator of disturbances in the well-being of the person being monitored. The blood sugar level can in turn be an indicator of whether a person being monitored is hungry.

According to an aspect of the invention, a system for monitoring a person, in particular for monitoring a sleeping infant, is created, comprising a monitoring device as described above and a washable item of clothing for the person. In this case, the sensor device can advantageously be connected both mechanically and electrically to the item of clothing, and when the sensor device is connected to the item of clothing, vital data of the person wearing the item of clothing are transmitted to the base station. The monitoring device with the sensor device can advantageously be connected to new or freshly washed items of clothing, such that the sensor device can be used for different items of clothing and people. The sensor device is expediently designed as described above.

According to an aspect of the invention, a method for monitoring a person, in particular for monitoring a sleeping infant, is created, using a monitoring device as described above, in which the sensor device measures the temperature and the oxygen saturation in the person's blood as well as at least one other vital parameter of the person, selected from the group comprising: body temperature, blood pressure, blood sugar level, wherein the sensor device transmits the measured vital parameters wirelessly to the base station, wherein the monitoring module uses a camera to capture an image of the person being monitored, wherein the base station uses a microphone to capture acoustic signals, wherein the base station shows the captured image and the vital parameters on a display of the monitoring module, and also transmits the same wirelessly together with the acoustic signals in a network for display on an external device.

According to an aspect of the invention, a method for monitoring a person, in particular for monitoring a sleeping infant, is created, using a monitoring device comprising a base station that can be supplied with power and that has a base and a monitoring module that can be pivoted with respect to the base; and a sensor device that can be connected to an item of clothing of the person, wherein the sensor device measures one or more vital parameters of the person, selected from the group comprising: heartbeat or pulse, body temperature, blood pressure, blood sugar level, and transmits the same wirelessly to the base station, wherein the monitoring module captures an image of the person being monitored by means of a camera, and wherein the base station captures acoustic signals by means of a microphone, wherein the base station displays the captured image and the vital parameters on a display of the monitoring module and also wirelessly transmits the same together with the acoustic signals in a network for display on an external device. In this case, the sensor device comprises a sensor for measuring the oxygen saturation in the person's blood as a first vital parameter, which is transmitted with the at least one of the selected further vital parameters.

The method advantageously enables the monitoring of a person, in particular a small child, because both sounds and images of the person are transmitted, as well as at least one and preferably several of the vital parameters mentioned. It is possible to classify the vital signs or parameters in terms of a range that is acceptable, a range that may be problematic, and a critical range, in order to clearly indicate an alarm situation to a user. Corresponding threshold values, which are compared with the vital parameters, are stored for the ranges. It is possible that the threshold values are preset by default, for example by entering the age of the person being monitored, and can be changed subsequently.

A monitoring device as described above and preferably an associated sensor device as described above are expediently used for the aforementioned method. The method expediently uses the sensor device on an item of clothing of the person and the base station in the vicinity of the person in a way making it possible to capture both the vital parameters captured from the sensor device and the image and sound. However, a user can call up the expediently processed data on an external device, in particular a tablet PC or a smartphone, with a correspondingly installed software application (app) and identification on the base station, and also control the base station. This advantageously enables monitoring the person even from a location remote from the person, for example the monitoring of several small children in a maternity ward from the room of a caregiver. The data from several monitoring devices can also be displayed on an external device. In the event of an alarm resulting from the vital parameters deviating from a permissible range, the caregiver can reliably check the correct person being monitored and, if necessary, undertake resuscitation measures or provide a drug administration.

According to an expedient implementation, it is provided that the base station contains an evaluation logic that determines an individual base temperature of the healthy person and accordingly automatically defines the areas for certain alarm states. Since the base temperature can be very different from person to person, a setting that prevents false alarms is created in this way.

It is possible to equip the sensor device with a vibration generator that, for example, wakes the person being monitored when certain vital parameters deviate from permissible ranges. In this way it can be achieved, above all, advantageously that if certain symptoms arise, for example a drop in oxygen saturation in the blood, the person is induced to change position in their bed, or is induced to start breathing. Additionally or alternatively, an acoustic signal can also be generated via the loudspeaker, similar to an alarm clock.

The base station can be integrated wirelessly or by wire into a hospital network, such that several base stations can be monitored in one or in several rooms at the same time. This can also be integrated into the digital hospital management system.

According to an expedient refinement, the microphone can be built into the sensor device, which can then also detect heartbeats acoustically. Furthermore, sounds made by a small child are captured particularly reliably.

The base station preferably also has further sensor means suitable for room monitoring. In addition to the microphone, this is, for example, a temperature measuring sensor and/or a room air humidity measuring sensor.

Further advantages, features, properties and developments emerge from the following description of a preferred embodiment and from the dependent claims. The invention is explained below with reference to the accompanying drawings using a preferred embodiment.

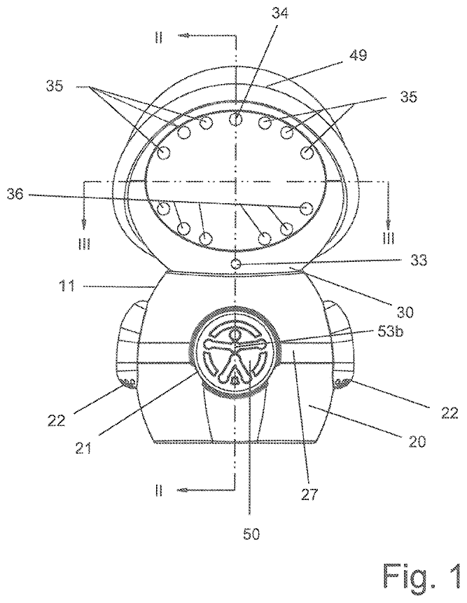

FIG. 1 is a view from the front of a preferred embodiment of a monitoring device according to the invention.

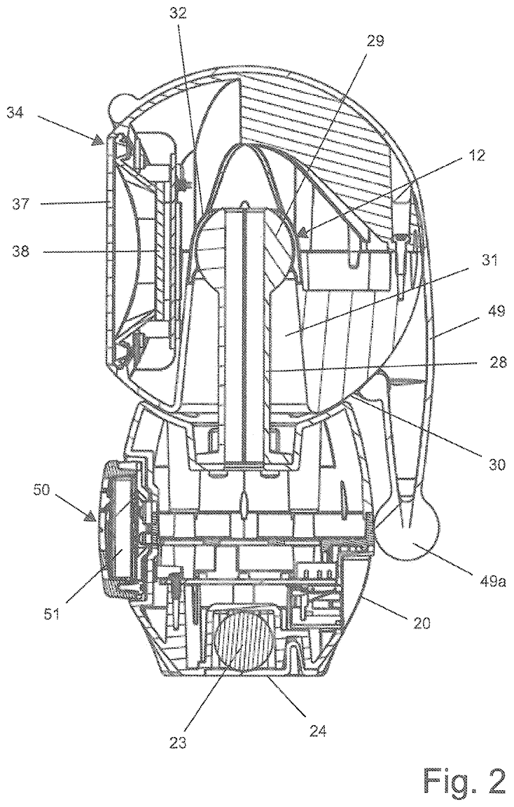

FIG. 2 is a longitudinal section through the monitoring device of FIG. 1, along the line II-II.

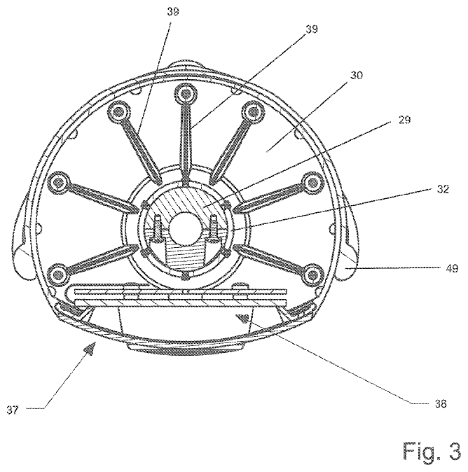

FIG. 3 is a cross section through the monitoring device of FIG. 1, along the line III-III.

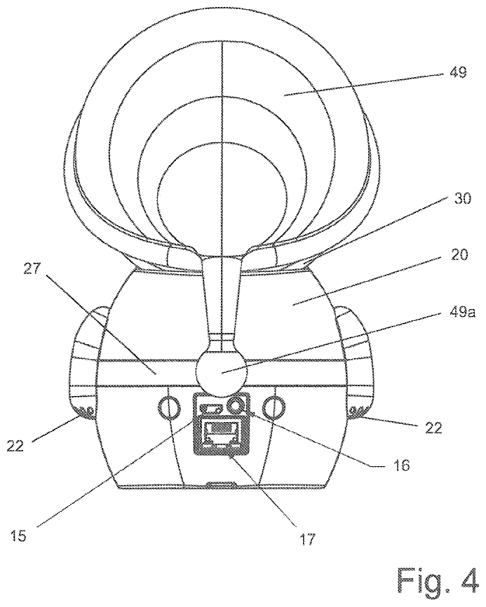

FIG. 4 is the monitoring device of FIG. 1 to 3, from behind.

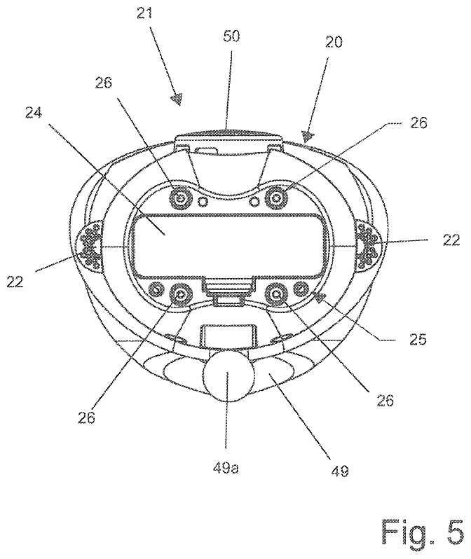

FIG. 5 is the monitoring device of FIG. 1 to 4, from below.

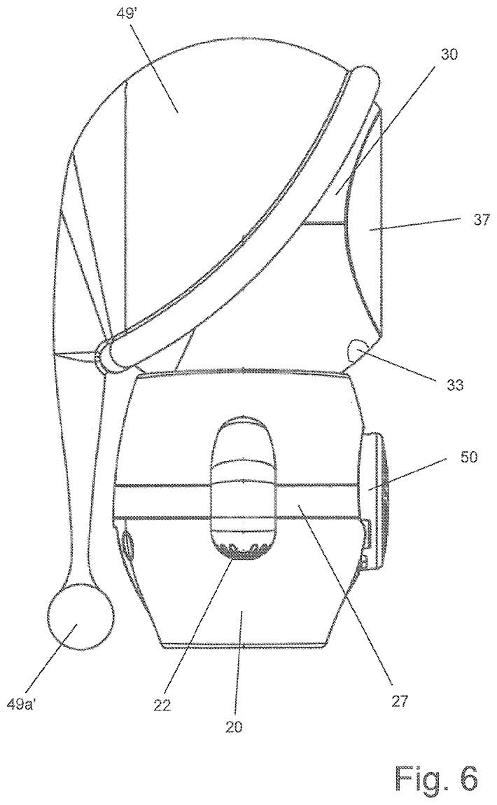

FIG. 6 is a modified monitoring device similar to FIG. 1 to 5, from the side.

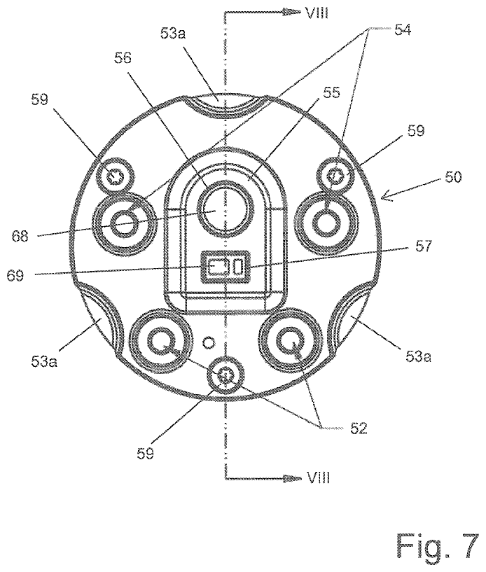

FIG. 7 is the sensor device of the monitoring device according to FIG. 1 to 6, from below.

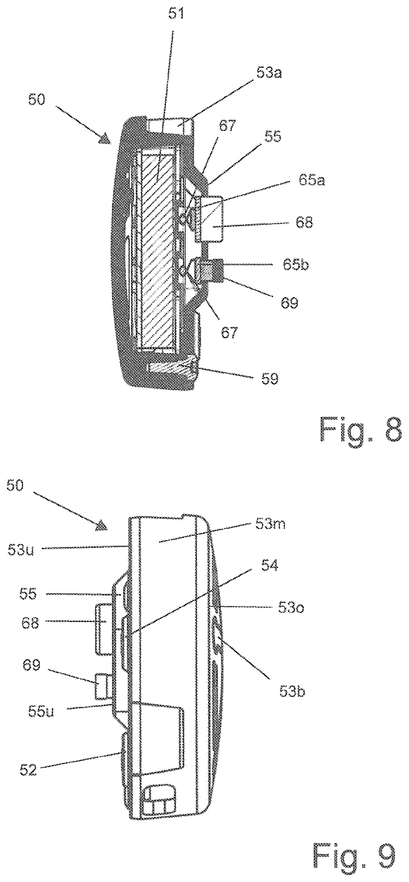

FIG. 8 is a section through the sensor device of FIG. 7, along the line VIII-VIII.

FIG. 9 is the sensor device of FIGS. 1 and 2, from the side.

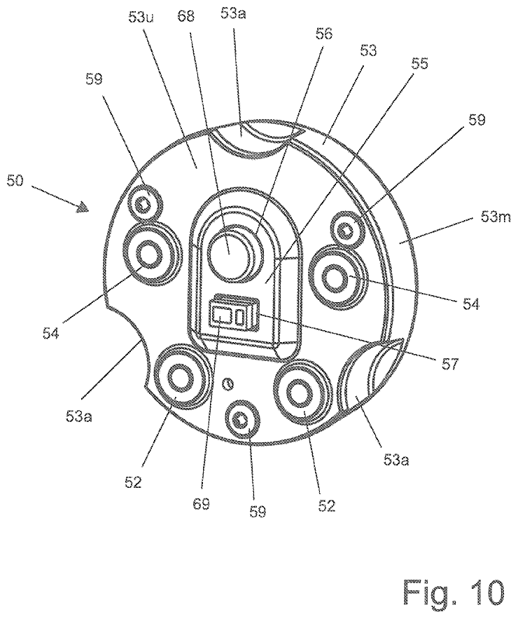

FIG. 10. is a perspective view of the sensor device of FIG. 7 to 9, from below.

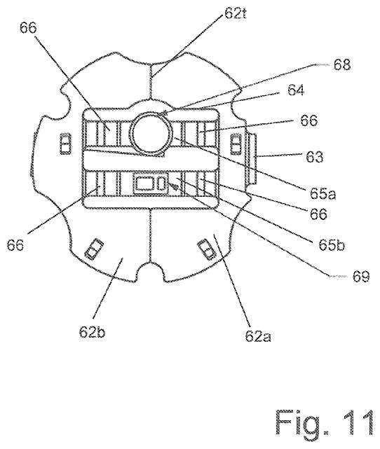

FIG. 11 is the sensor device of FIG. 7 to 10, from below, without a housing.

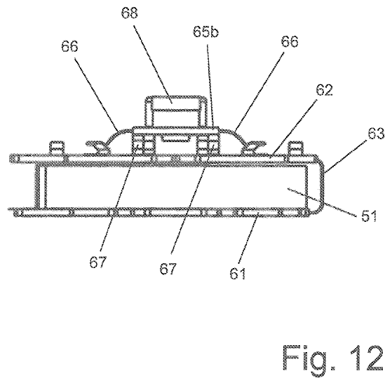

FIG. 12 is the sensor device of FIG. 11, from the side.

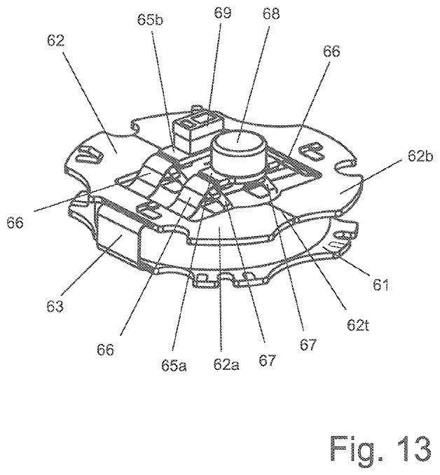

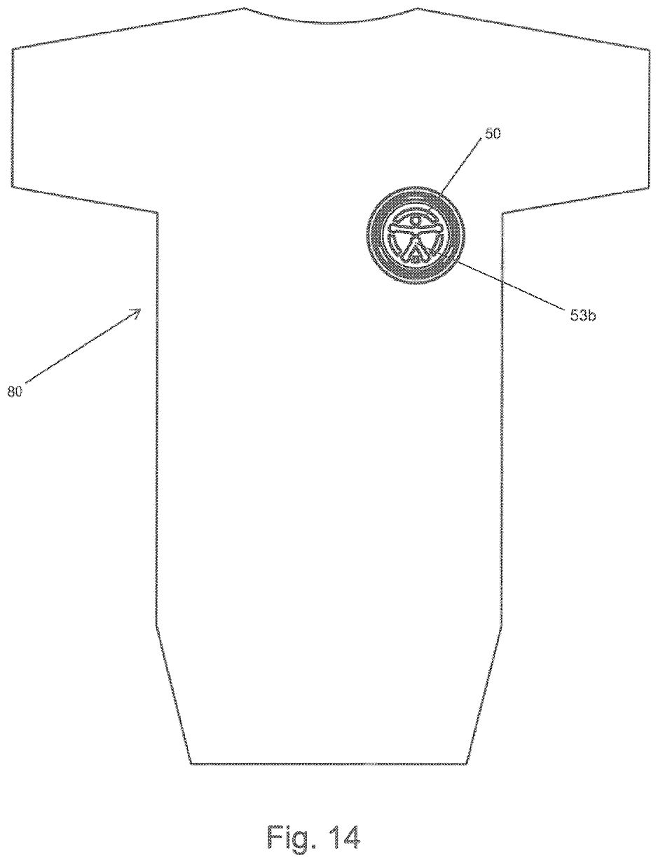

FIG. 13 is the sensor device of FIGS. 11 and 12, in perspective from below.

FIG. 14 is the outside of an item of baby clothing, with a connected sensor device, from the front.

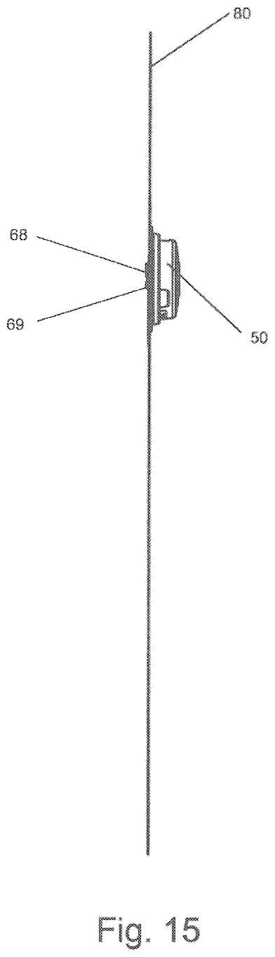

FIG. 15 is the item of baby clothing of FIG. 14, with the connected sensor device, from the side.

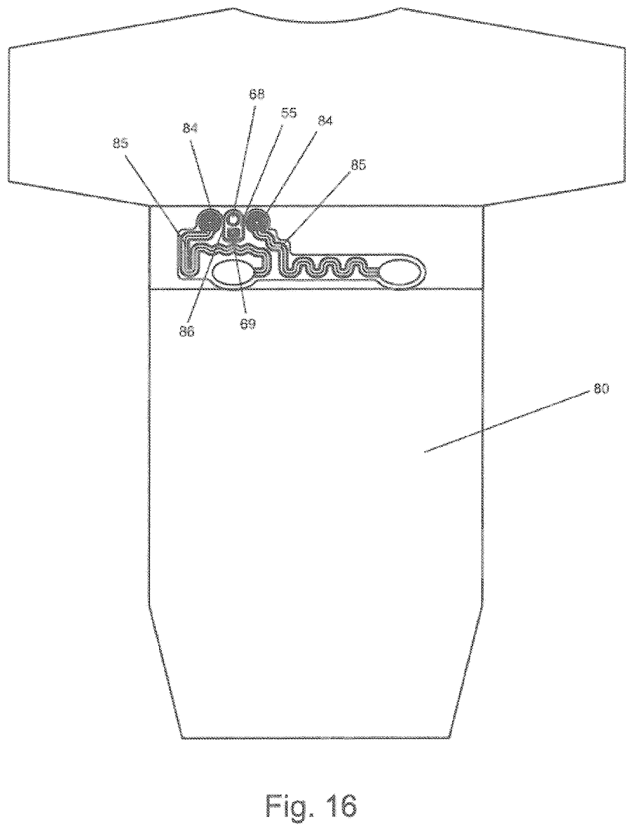

FIG. 16 is the inside of the item of baby clothing of FIGS. 14 and 15, with attached contacts.

FIG. 1 is a preferred embodiment of a monitoring device 10 according to the invention, from the front. The monitoring device 10 has the silhouette of a human infant, in which a relatively large head is formed on a trunk of approximately the same size. The monitoring device 10 comprises a base station 11, which in turn comprises a base 20 corresponding to the body and a monitoring module 30 corresponding to the head. A sensor device 50, which will be described in more detail below, is connected to the base at the maximum circumference of the base 20.

The base in this case is equipped with a charging station 21 in which a rechargeable power source 51 of the sensor device 50, designed as a lithium ion battery, can be charged. The charging station forms a recess in the body of the base into which the sensor device 50 can be inserted and in which the sensor device 50 is held by clamping. The charging takes place with contacting, that is charging contacts 52 of the power source 51 are contacted in the charging station 21. The corresponding charging contacts of the charging station 21 can be designed like push buttons in order to avoid loose contacts, and at the same time reliably fix the sensor device 50 in the charging station 21.

On both of its sides, the base has arm-like extensions that are each designed with a loudspeaker 22 on their lower ends. A battery 23 is also built into the base 22 as an emergency power supply. The access to the battery 23, in order to be able to exchange it, takes place via a flap 24 in the foot of the base 20 (see FIG. 5).

The base has a fairly low center of gravity so that it does not tend to tip over. In addition, fixing means are also provided in the foot of the base 20, which in the present case are designed as a built-in neodymium magnet 25, such that magnetic fixing on a magnetizable base is made possible. In addition, four screw holes 26 are provided as alternative fixing means in the foot of the base 20, with which the base 20 can also be fixed to a base provided with corresponding screws.

At its approximately maximum circumference, that is to say at the level of the charging station 21, the base 20 has a completely or at least predominantly circumferential lighting arrangement that is designed as a luminous ring 27 illuminated by LEDs. The LEDs are dimmable and can be used as a night light. At the same time, the color of the illuminated ring can be used as a display for alarm conditions, for example in the same way as the color of a traffic light in traffic.

It can be seen in FIG. 4 that the base provides various contacts on its rear side. On the one hand, there is a power supply input designed as a USB connection 15. This means that the base can be connected to a wide variety of external power sources for the supply of power. In addition to the USB connection 15, a jack 16 is provided as an audio output. Under the two contacts mentioned, an Ethernet socket 17 is provided, which provides data input and output, enabling wired communication as an alternative to wireless communication of the data. Software updates in particular can be installed reliably and conveniently.

It can be seen in particular in the illustration according to FIG. 2 that the base station 11 enables the monitoring module 30 to be pivoted relative to the base via a ball joint 12 of the base station 11. At its upper end facing away from the foot, the base 20 has an upwardly projecting (hollow) cylindrical portion 28, at the end of which a ball 29 is formed. At its lower end, the monitoring module has a conical recess 31 on which a ball socket 32 receiving the ball 29 is formed. The ball joint 12 makes it possible to rotate the monitoring module 30 relative to the base 20, and also to adjust the inclination of the monitoring module 30 relative to the base 20, wherein the conical recess 31 limits the adjustment angle. The monitoring module 30 can thus be pivoted in several directions with respect to the base 20. The conical recess 31 is dimensioned in this case in such a way that even with maximum pivoting the recess 31 is not open to the surroundings unprotected.

The monitoring module 30 is covered in an area corresponding to the back of the head by a cap 49 made of textile material. The cap 49 is lengthened approximately at the height of the luminous ring 27, at the level of which a pointed end 29a is provided. The cap 49 is mainly used for decoration. In the alternative embodiment according to FIG. 6, however, the tip 29a′ of the cap 29′ continues to the level of the foot of the base 20, and the material of the cap is selected for the transmission of compressive forces in such a way that the cap 29′ forms an additional support for the base station 10, in the manner of a third leg.

The design of the monitoring module 30 approximates that of a human face. Here, the monitoring module 30 has a microphone 33 that can detect acoustic signals from the room being monitored. The monitoring module 30 also has a camera 34 that can be pointed at a person being monitored by pivoting in the region of the ball joint 12. Arranged adjacent to the camera 34 are infrared bulbs 35 that illuminate the person being monitored without generating a perceptibly brighter visible light in the process. Further recesses 36 are provided to either accommodate further infrared bulbs 35 or to accommodate signal LEDs that are used as indicators for various operating or alarm states.

A region of the monitoring module 30 corresponding to the eye area is covered by a curved, transparent pane 37 made of a transparent material, in the present case glass, which has a touch functionality. The pane 37 can also be made of transparent plastic, but this makes it sensitive to scratching. The pane 37 does not cover the camera 35, the IR bulbs 35 and the recesses 36; however, it is also possible to design the pane 37 in such a way that the corresponding elements are covered and thus protected. Due to the touch functionality, the pane 37 serves as an integrated input unit via which control commands can be input to the monitoring device 10. This avoids interrupting the rounded optics of the monitoring device 10. In the present case, the curvature of the pane 37 is in only one dimension, in the region of its lateral ends; it is also possible to provide a further curvature in the vertical dimension.

In the interior of the monitoring module 30, behind the transparent pane 37, a flat display 38 is arranged, which in the present case is oriented vertically. Images and data can be displayed on the display 38, in particular the measurement data that the sensor device 50 captures, as well as information derived therefrom. The sensitive display 38 is thus arranged in such a way that it is protected from access by a user. The display 38 must therefore be viewed through the pane 37, as can be seen in particular in FIG. 3. The display 38 and the touch-sensitive pane 37 together provide a functionality comparable to a smartphone display.

It can also be seen in FIG. 3 that the ball socket 32 is held on the monitoring module 30 by struts 39.

FIG. 7 to 10 show the sensor device 50 in more detail. A housing 53 encapsulates the sensor device 50 and has a convex, outwardly curved upper side 53o that points forwards when inserted into the charging station 21, and a substantially flat lower side 53u, which provides the charging contacts 52. The housing 53 is designed essentially in the manner of a disk without much thickness, wherein the circumferential shell 53m has three recesses 53a in the region of its transition to the lower side 53u, which allow the housing to be ergonomically easier to grip when it is removed from the charging station 21. On the upper side 53o, the housing has a representation of a figure, which is implemented by illuminable portions 53b. The illuminable portions 53b, which can be clearly seen in FIG. 1, form a display 53b for the state of charge of the battery 51, based on the colors of a traffic light.

It can be seen in FIGS. 7, 8 and 10 that two sensor points 54 are designed as pushbuttons, which can detect heartbeat and breathing when they are connected to a correspondingly designed garment 80, as shown in FIG. 14 to 16, for example. It can be seen that the sensor points 54 are clipped into corresponding contacts 84 of a conductor path 85 attached to the inside of the garment 80, and are thus permanently connected to the garment 80, in the present case a baby shirt. It can also be seen that between the two contacts 84 a recess 86 is formed in the garment 80, which is intended for the passage of further sensors housed in the sensor device, which then rest directly on the baby's skin in the baby's chest area. The item of clothing 80, including the conductor paths 84, can be washed and reused while the sensor device 50 is removed from the item of clothing 80 and placed back in the charging station 21.

It can be seen in FIGS. 7 and 10 that the distances between the charging contacts 52 and the sensor points 54 are configured to be different, in order to prevent incorrect operation. It can also be seen in FIG. 7 to 10 that the housing 53 has on its underside 53u in a central portion a protruding region 55 that occupies an area that corresponds to a square topped by a semicircle. The underside 53u transition via rounded edges into a plane 55u parallel to the underside, wherein two openings, an upper round opening 56 and a lower rectangular opening 57, are cut out in the plane 55u. Through the openings 56, 57, further sensors, which will be explained in more detail below, and for which the recess 86 is also provided, are pressed against the skin of the person being monitored.

Finally, three screws 59 can be seen which penetrate the housing 53 from the underside 53u and which can be removed to dismantle the housing 53.

The inner workings of the sensor arrangement 50, and thus the actual electronics assembly 60, are shown without the housing 53 in FIG. 11 to 13. It can be seen that the power source 51 is arranged in a sandwich between an upper board 61 and a lower board 62, which is formed by a first base board 62a and a second base board 62b. The upper board 61 faces the upper side 53o; the lower board 62 faces the lower side 53u. The circuit boards carry additional components that are not shown in detail and that can also be implemented as microchips. The upper circuit board 61 and the lower circuit board 62 are connected to each other via a flexible cable or flexible circuit board element 63, which provides conductor paths for the components.

In particular, the electronic assembly 60 comprises a Bluetooth transmitter, which enables a wireless connection to a receiver arranged in the base station 11 for the exchange of data. A wireless LAN transmitter is also accommodated in the base station 11, which enables a wireless connection to an external device such as a smartphone.

The two base boards 62a, 62b are separated from each other along a separating line 62t. In their center they bound a recess 64 in which two lift boards 65a, 65b are arranged. The lift boards 65a, 65b are designed as rectangular board pieces, each of is connected by means of a flexible board element 66 to the base board 62a on one end and to the base board 62b on the other end. This connection also constitutes the electrical connection of the two base boards 62a, 62b. The two lift boards 65a, 65b are biased by a spring arrangement 67 in the direction away from the two base boards 62a, 62b, wherein sensor arrangements 68, 69 arranged on each of the lift boards 65a, 65b are biased towards the skin of a person being monitored. The spring arrangement 67 comprises two spring elements arranged in opposite directions, which bias the lift board 65a, 65b into a raised position. As such, it is only when the two base boards 62a, 62b are pushed together, until they meet in the region of the dividing line 62t, that the lift boards can be raised. The spring arrangements 67 passing through the recess 64 then ensure that the sensors 68, 69 are not pushed away from the skin of the person being monitored.

The sensor 68 is designed as a contact sensor that detects the surface body temperature of a person; on the basis of this, the body core temperature can be reliably determined with the corresponding information about the point at which the surface body temperature was captured. It is important that the temperature sensor 68 is always in contact with the person's skin.

The sensor 69 is designed as a sensor for measuring the oxygen saturation in the person's blood. It comprises a light transmitter that emits light into a surface layer of the body and a receiver that detects light reflected in the surface layers, and from this reliably calculates the oxygen saturation in the person's blood. For this purpose, several light transmitters in the form of LEDs can also be provided, which emit optionally pulsed and thus coded signals that the receiver evaluates. Here, too, it is important that as far as possible no changes in the distance between the sensor and the person's skin occur.

In order to ensure optimal tracking of the sensors 68, 69, both are arranged on their own lift board, which lift boards can react individually to movements of the person.

The invention then works as follows:

In order to monitor a person, in particular a sleeping infant, the latter is first dressed in an item of clothing 80 that enables the sensor device 50 to be connected via the push-button contacts 54. The monitoring device 10 is used to monitor the person, the base station 11 being positioned in such a way that when the monitoring module 30 is appropriately pivoted about the ball joint 12 with respect to the base 20, the person being monitored is in the field of view of the camera 34.

The sensor device 50 is initially located in the charging station 21 of the base station 11, and its power source 51 is charged there. The charge status of the power source 51 of the sensor device can be recognized via the display 53b. It is possible to additionally provide a display for the state of charge of the power source 51 of the sensor device 50 on the base station. If the power source 51 has a sufficient charge, the sensor device 50 can be removed from the charging station 21 and connected to the item of clothing 80.

The sensor device 50 has a Bluetooth transmitter that communicates with an associated receiving module of the base station 11 and wirelessly transmits the vital data or the vital parameters of the person, which are captured by the corresponding sensors. In the present embodiment, the vital data are oxygen saturation in the person's blood, heartbeat or pulse of the person, and the person's body temperature. However, other vital parameters such as blood pressure, blood sugar level, systole, diastole and the like can also be captured directly or indirectly by a sensor and transmitted wirelessly to the base station. In particular, the wireless transmission prevents damage to the lines, caused by kinks, from triggering false alarms.

Sounds in the room in which the person is being monitored are captured by means of the microphone 22. Both the image or video data captured by the base station 11 and the acoustic data, and also the vital parameters captured by the sensor device 50, are transmitted by a transmitting device in the base station, for example a wireless LAN transmitter equipped with a microprocessor, or by wire via the provided USB socket 15, to a network, in particular on the Internet, such that an image or a film of the person being monitored, and/or processed or unprocessed vital parameters of the person and the captured acoustic signals, are transmitted. It is possible to also have this information shown on the display 38, in which case the same image is expediently displayed and the sound is reproduced via the loudspeaker 22 as in an external operating device such as a smartphone. If certain vital parameters are outside a normal range, the corresponding information is highlighted with signal colors or signal sounds in order to enable the monitoring caregiver to intervene.

The invention has been explained above using an embodiment in which a flat display 38 for displaying information is arranged in the monitoring module 30. It has to be understood that other display devices with a display can also be used for this purpose, the display 38 preferably being arranged inclined to the vertical so that a caregiver looking through the transparent pane 37 from above can read the data easily and reliably.

The invention has been explained above using an embodiment in which the base station has the silhouette of a small person. It has to be understood that the external design of the base station can also be configured completely differently, and that in particular the position of the microphone, camera, IR bulbs, LED and loudspeaker can also be provided at other points on the base station.

The invention has been explained above on the basis of an embodiment in which the pivotability of the monitoring module 30 relative to the base 20 has been implemented via a ball joint 12. It has to be understood that other articulated connections are also possible between these two parts 20, 30, also with the interposition of further elements that space the monitoring module 30 apart from the base 20. In particular, it can also be provided that several joints or a telescope provide greater flexibility.

The invention has been explained above on the basis of an embodiment in which a single sensor device 50 is assigned to the base station 11, as a result of which the monitoring of the person has to be repeatedly interrupted for charging purposes in the charging station 21. It has to be understood that the base station 11 can also be assigned more than one sensor device 50, for example two or more sensor devices 50, these then being in data connection with the base station 11 so that either the one sensor device 50 in use on an item of clothing 80 and the other sensor device 50 can be arranged in the charging station 21, or different parameters can also be detected by different sensors in the sensor device 50, depending on the specific monitoring requirement.

The invention has been explained above on the basis of an embodiment in which the sensor device 50 has two lift boards 65a, 65b with different sensors 68, 69. It has to be understood that the sensor device 50 can also have only one sensor provided on a lift board, or else more than two lift boards with sensors. It is also possible to accommodate more than one sensor on one board, or the same sensor on two different lift boards for redundant measurement, to increase safety.

The invention has been explained above on the basis of an embodiment in which the sensor device 50 is charged in a charging station 21 formed in the base 20. It has to be understood that the charging station can also be provided on another part of the monitoring device 10, or that the sensor device 50 can be supplied with power in a wired manner. The particular advantage of the charging contacts 52, however, is that the housing 53 can be completely encapsulated so that liquids cannot penetrate into the housing 53.

1. A monitoring device for a person, in particular for monitoring a sleeping infant, comprising
a base station that can be supplied with power, and comprising a base and a monitoring module that can be pivoted with respect to the base; and
a sensor device that can be connected to an item of clothing of the person,
wherein the sensor device comprises a rechargeable power source that supplies the sensor device with power,
wherein the sensor device comprises a sensor for measuring the oxygen saturation in the blood of the person,
wherein the sensor device for detecting one of the heartbeat and the pulse of the person,
wherein the base station has a charging station for the rechargeable power source of the sensor device,
wherein the sensor device is wirelessly connected to the base station for transmitting the vital data measured by the sensors,
wherein the monitoring module of the base station comprises a camera that can be pointed at the person by pivoting the monitoring module with respect to the base,
wherein the monitoring module comprises an input unit and a display unit,
wherein the input unit is designed as a curved, transparent pane with touch functionality, and
wherein the display unit is designed as a flat display arranged behind the transparent pane.

2. The monitoring device according to claim 1, wherein the base station comprises a microphone that detects acoustic signals, in particular from the person being monitored and wherein the base station comprises a loudspeaker that can generate prespecifiable acoustic signals.

3. (canceled)

4. The monitoring device according to claim 1, wherein the monitoring module comprises at least one infrared bulb, and wherein the base station comprises a lower base surface with a fixing arrangement for attaching the base station to an external surface.

5. (canceled)

6. The monitoring device according to claim 1, wherein the base station comprises at least one lighting arrangement that is dimmable, and wherein the base station comprises LEDs of different colors for displaying alarm states.

7. The monitoring device according to claim 1, wherein the display can display the captured vital data, and wherein the curved transparent pane is selected from the group consisting of glass and plastic.

8. The monitoring device according to claim 1, wherein the base station is modeled on the silhouette of an infant, wherein the base corresponds to the trunk and the monitoring module corresponds to the head of the infant, and wherein the curved transparent pane essentially covers an eye area of the head.

9. The monitoring device according to claim 1, wherein the base station is supplied with power by alternating current and has a charging station for the power source of the sensor device, which can be repeatedly charged with direct current, and wherein at least one of the base station and the sensor device has a display of the state of charge of the rechargeable power source of the sensor device.

10. The monitoring device according to claim 1, wherein the charging station is provided in the base, and wherein the sensor device is insertable into the charging station by clamping.

11. (canceled)

12. The monitoring device according to claim 1, wherein the power source has charging contacts designed as push buttons that can be connected to the charging station by clamping.

13. (canceled)

14. The monitoring device according to claim 1, wherein the base station comprises a wireless transmitter that provides digital data, in particular measured vital data, in a network.

15. The monitoring device according to claim 1, wherein the monitoring module is connected to the base by means of a ball joint that enables pivoting.

16. The monitoring device according to claim 1, wherein the sensor device comprises a housing in which are arranged the power source and a transmission arrangement for wireless transmission of vital data captured by the sensors to an external receiving station.

17. The monitoring device according to claim 12, wherein the housing includes a connector for connection to an item of clothing of a person being monitored.

18. The monitoring device according to claim 13, wherein the connector comprises sensor points in the form of push buttons, which enable both a mechanical connection to the item of clothing and an electrical connection to a contact arranged on the item of clothing.

19. The monitoring device claim 1, wherein a sensor of the sensor device is arranged on a lifting board, wherein the lifting board is connected by flexible board elements to a first base board and to a second base board, and wherein the lifting board can be moved into a raised position by means of the flexible board elements and a spring arrangement, loading the lifting board upwards, by pushing the first base board and the second base board together.

20. A monitoring device for a person, in particular for monitoring a sleeping infant, comprising:
a base station that can be supplied with power; and
a sensor device that can be connected to an item of clothing of the person,
wherein the sensor device comprises a sensor device housing in which a rechargeable power source is arranged, which supplies the sensor device with power,
wherein the sensor device housing comprises a connector for connecting the sensor device housing to the item of clothing,
wherein the sensor device comprises at least one sensor that is biased in a direction of the person being monitored by a spring arrangement, wherein the at least one sensor is configured to capture vital data of the person,
wherein the sensor device comprises a transmission arrangement that wirelessly transmits the vital data captured by the at least one sensor to an external receiving station,
wherein the sensor is arranged on a lifting board,
wherein the lifting board is connected by flexible board elements to a first base board and to a second base board, and
wherein the lifting board can be moved into a raised position by means of the flexible board elements and the spring arrangement by pushing the first base board and the second base board together.

21. (canceled)

22. The sensor device according to claim 16, wherein a first and a second sensor are provided, and wherein the first and the second sensor are arranged on mutually parallel, independently spring-loaded lifting boards.

23. (canceled)

24. The sensor device according to any claim 17, wherein the sensor device comprises a vibration generator for waking the person.

25-28. (canceled)

29. A system for monitoring a person, in particular for monitoring a sleeping infant,
comprising
a monitoring device; and
a washable garment for the person; and
a sensor device including a rechargeable power source that supplies the sensor device with power;
wherein the monitoring device comprises a base station that can be supplied with power,
wherein the base station includes a charging station for the rechargeable power source of the sensor device,
wherein the base station comprises a camera that can capture video data of the person,
wherein the base station comprises a microphone that can capture audio data of the person,
wherein the base station comprises an input unit and a display unit,
wherein the sensor device comprises at least one sensor for measuring vital data of the person,
wherein the sensor device is wirelessly connected to at least the base station for transmitting the vital data measured by the sensors,
wherein the sensor device can be repeatably connected to and removed from the washable garment,
wherein the sensor device can be repeatably connected to and removed from the charging station of the monitoring device for loading the rechargeable power source, such that when the sensor device is connected to the washable garment worn by the person, vital data of the person wearing the washable garment are measured by the sensor and transmitted at least to the base station.

30. The system according to claim 19, wherein the sensor is selected from a group comprising: a sensor for measuring the oxygen saturation in the person's blood, a sensor for detecting one of the heartbeat and the pulse of the person, a sensor for detecting the surface body temperature of the person, a sensor for detecting the blood pressure of the person, a sensor for detecting the blood sugar level, and combinations thereof.
